(19) United States
(12) Patent Application Publication
(10) Pub. No.: US 2017/0145367 A1
(43) Pub. Date: May 25, 2017

(54) IN VITRO BIOSIMULATOR TO INDUCE PATTERN FORMATION IN NON-ADHERENT CELLS

(71) Applicant: Sunil Thomas, Philadelphia, PA (US)

(72) Inventor: Sunil Thomas, Philadelphia, PA (US)

(21) Appl. No.: 14/948,272

(22) Filed: Nov. 21, 2015

(51) Int. Cl. C12M 1/12

(52) U.S. Cl. CPC C12M 25/06

(57) ABSTRACT

It is not understood what causes or influences pattern formation in cells during the development of an organism. When animal/human cells are cultured in a Petri dish the adherent cells attach to the bottom of the dish, whereas the non-adherent cells float in the growing medium. Currently there are no specialized dishes for culturing non-adherent cells. We now show that non-adherent cells could be induced to form distinct patterns when cultured in an etched plastic dish (Biosimulator). The non-adherent cells showed polarity when cultured in the etched plate. The polarity/pattern formation could be reversed with inhibitors specific for adhesion proteins. The phenomenon of pattern formation by non-adherent cells has wide applications in cell and developmental biology, diagnostics, microbiome research, biofluidics, drug discovery, industrial production of biological products, and also in biotechnology and bioengineering.

### BACKGROUND

1. Field of the Invention

The present invention relates generally to the fields of Cell Biology, Immunology, Microbiology, Pathology, Molecular Biology, Pharmacology, Biotechnology, and Bioengineering. Specifically, the present invention relates to the development of a biosimulator to induce pattern formation in non-adherent cells.

2. Description of the Related Art

Natural systems exhibit an amazing diversity of patterned structures in living systems such as animal coats (Wang et al. 2014). Alan Turing (1952) proposed a reaction-diffusion model explaining potential mechanism for animal coats: at a certain stage of embryonic development, the reaction and diffusion between molecules, known as morphogens, and other reactors, lead to the breaking of symmetry of the homogeneous state. The morphogens spontaneously evolve to a non-uniform state, leading to the unique textures seen on animal skin. As yet it is not known whether non-adherent cells form patterns in an organism.

Single-cell analysis provides information critical to understanding key disease processes that are characterized by significant cellular heterogeneity. Few current methods allow single-cell analysis without removing cells from the context of interest, which not only destroys contextual information but also may perturb the process under study (Sarkar et al. 2014). When adherent cells are cultured on a Petri dish it spreads rapidly and forms confluence within a couple of days. The time required to form confluence depends on the nature of cell (cell line). However, when non-adherent cells are cultured some cells attach to base of the Petri dish but the majority of cells are suspended in medium. As yet there are no specialized plates for culturing non-adherent cells. We now show that the non-adherent cells could be made to form distinct patterns when cultured on specialized etched plate (Biosimulator). The pattern formation of non-adherent cells has wide applications in biomedical research.

The prior art is deficient in inducing pattern formation in non-adherent cells. The present invention fulfils the long standing need and desire in the art.

### SUMMARY OF THF INVENTION

Embodiments described herein demonstrate a biosimulator with an etched surface to culture non-adherent cells. Culturing non-adherent cells to form distinct patterns on an etched surface has wide applications in biotechnology and bioengineering.

Certain embodiments are direct to an in vitro system comprising an etched plate where non-adherent cells can be cultured to form distinct patterns. In certain aspects the non-adherent cells can be microorganisms including bacteria, fungi, virus, phytoplasma, mycoplasma, or cells including B cells, T cells, neutrophils, red blood cells, hybridomas, monocyles/macrophages, etc. In other aspects the non-adherent entity will include organelles from cells including, chloroplast, mitochondria, ribosome etc. In other aspects the biosimulator can comprise an etched surface and any non-adherent cells that does not form adherence on conventional Petri dishes.

Other embodiments are directed to methods for designing biofouling resistant probes for bioengineering applications. Patterns of probes used for biomedical applications can be etched on a biosimulator. Culture of the non-adherent cells in the biosimulator will determine the affinity of cells on a particular pattern which will assist in developing probes and devices that resist biofouling.

Further embodiments include development of drugs that could modulate the non-adherent cells. Lack of adherence by non-adherent cells limits use of these cells in pharmacological studies. Use of the biosimulator will encourage development of novel drugs for non-adherent cells which are important in diseases including: autoimmune diseases (including type I diabetes, arthritis, etc), cardiac diseases, cancer, infectious and parasilic diseases. The biosimulator could also substitute for animal models, since drugs could be used to prevent adherence of cells and the data obtained with 5 days.

As used herein, “subject” refers to cells cultured in a biosimulator. In certain embodiments the subject is a human cell. In certain embodiments the subject is either a human, animal or microbial cell.

Other embodiments of the invention are discussed throughout this application. Any embodiment discussed with respect to one aspect of the invention applies to other aspect of the invention as well and vice versa. Each embodiment described herein is understood to be embodiments of the invention that are applicable to all aspects of the invention It is contemplated that any embodiment discussed herein can be implemented with respect to ally method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Pattern formation in non-adherent cells. A. Photomicrograph of non-adherent cells exhibiting polarity on an etched biosimulator. Non-adherent cells adhere above the etched line on the top half of the biosimulator, whereas they adhere below the etched line on the bottom half of the biosimulator. B. Drawing of an etched biosimulator showing the orientation of non-adherent cells.

FIG. 2. Pattern formation of non-adherent cells on different etched surfaces.

FIG. 3. Treatment of non-adherent cells with specific drugs prevented adhesion of cells to the etched plastic surface. A. Untreated cells, B. Salicylic acid treated cells, C Pectasol treated cells.

### DETAILED DESCRIPTION OF THE INVENTION

Conventional methods of cell culture include seeding of cells on Petri dishes. Cell culture treated dishes are used to grow adherent cells, where they are attached to the bottom of the Petri dish, whereas non-adherent cells do not attach to the dish. Non-adherent cells (including B cells, T cells, hybridomas) are suspended in the medium (Schindler, 1969).

Most of the bacterial culture uses agar as a solid medium. The bacterial cells growing on the semi-solid agar form distinct colonies, which are later used for several studies. However, only less than 1.0% of the bacteria are culturable. The ability to culture the majority of bacteria has impeded studies on new natural products and also has prevented factors that can contribute to both ecological balance and host health (Stewart, 2012).

In vitro cell culture is the first step to test the efficacy of pharmacological drugs. There are no existing technologies for the culture of non-adherent cells thereby impeding studies on drugs targeting these cells. A technique to make the non-adherent cells adherent to a surface will lead to development of novel drugs that impact diseases like type I diabetes, arthritis, allergy etc. (Rello et al. 2005).

In vitro culture is also used in the culture of organelles like choloroplast (Leiva-Mora et al. 2010) for the synthesis of sugar or glucose for biofuel production.

In vitro tissue culture is also used in the production of meat (van Eelan, 2007). The meat thus produced as an impact on the environment as lower amount of food and water are required to raise livestock. The carbon emission is also lowered when meat is cultured in vitro.

Recently 3D cell culture has gained popularity. A 3D cell culture is an artificially-created environment in which biological cells are permitted to grow or interact with their surroundings in all three dimensions. This is an improvement over the previous method of growing cells in 2D (on a Petri dish) because the 3D model more accurately models the in vivo cells. These three-dimensional cultures are usually grown in bioreactors, small capsules in which the cells can grow into spheroids, or 3D cell colonies (Haycock, 2011; Ravi et al. 2015). However, the 3D cultures are not suitable for non-adherent cells.

Cell culture in Petri dishes is the first step in the production of novel biological products, or for identification of microorganisms, testing the efficacy of new pharmaceutical drugs, etc. As yet there are no in vitro systems for the culture of non-adherent cells. This led to the development of a new culture dish (biosimulator) that induces pattern formation in non-adherent cells. The method to design the biosimulator to induce pattern formation in non adherent cells is described herein.

Embodiments described herein demonstrate an in vitro system (biosimulator) for the culture of non-adherent cells. The in vitro system is an etched plastic surface. The non-adherent cells form distinct patterns after culture in the in vitro biosimulator. The non-adherent cells form distinct patterns based on the etching design.

The present invention provides a mechanism by which non-adherent cells can form distinct patterns on modified plastic surfaces. The present invention provides mechanism by which the pattern formation of non-adherent cells could be altered. Thus, in one aspect the present invention provides an efficacious mechanism to induce pattern formation in non-adherent cells.

The present invention also provides a mechanism by which the non-adherent cells form polarity in in vitro culture. The non-adherent cells when cultured on an etched plate (design: parallel lines), adhere on top of the line on the upper part of the dish, whereas, the cells are attached below the line in the lower part of the dish.

Non-adherent cells of the present invention include cell and/or cell lines. Examples of such cells and cell lines include primary cells (e.g., monocytes, T cell, B cell, RBC) and/or cell lines/continuous cell lines such as hybridomas that are non-adherent.

A cell culture may be grown in flasks, and subsequently passed to larger flasks to obtain larger volumes of material required to make cell lines. Alternatively, the infected cell culture may be passed from flasks into subsequent roller bottles, spinner flasks, cell cubes, bioreactors, or any apparatus capable of growing cell culture on large scale in order to produce a suitable quantity of material. Cell cultures may be frozen down in a suitable media and used for cell culture later.

In certain aspects of the present invention, the 10 cm biosimulator is seeded with 200, 000 to 400,000 cells. The cells are counted by a hemocytometer or any other electronic cell counter. The non-adherent cells form patterns 3-4 days after cell culture.

The non-adherent entity could also be organelles like chloroplast, mitochondria, and ribosomes. The organelle culture could induce generation of bio-products like glucose, sugar, proteins, etc.

The non-adherent entity could also be microorganisms including bacteria, virus fungi and parasites. Only a small fraction of the microorganisms are culturable. The etches of a biosimulator facilitates growth of non-culturable microorganisms. The strategy could be used to identify microbiome of animals or for the culture of non-culturable animal and plant pathogens.

In some embodiments, animal cells or cell lines including hybridomas (in a biosimulator) are typically grown at 37° C., in the presence of 5% CO₂. Microorganisms can be cultured with or without CO₂, at varying temperatures.

The pattern formation of non-adherence cells in a biosimulator can be observed by microscopy. The pattern formation of cells in a biosimulator can be observed with or without chemical dyes or stains.

Etches are made on the plastic surface with steel blades or lasers or any other material that could form the etching pattern. The etching pattern could also be modified using nano-materials like graphene.

Biofouling is an undesirable growth of microorganisms on probes or medical devices. Currently biofouling is prevented by using specialized coatings. The present invention could predict the areas of the probes or devices susceptible to biofouling by etching the pattern on the biosimulator. Based on the growth of the cells on a particular pattern, the probes or devices could be designed so that biofouling could be prevented.

In cardiovascular diseases, the white blood cells (WBCs) including monocytes can block the arteries. Culturing of WBCs from patients susceptible to cardiovascular diseases in a biosimulator could predict early diagnosis of the disease. The

The malaria parasite, Plasmodium resides in the red blood cells. Recent studies by Lu et al (2008, 2010) demonstrated that RBCs are culturable. The biosimulator could be used to diagnose Plasmodium infected RBCs.

In certain embodiments the efficacy of pharmaceutical drugs on non-adherent cells could be studied using a biosimulator. Those drugs that prevent adhesion of the cells can be identified. We had demonstrated that drugs that inhibit adhesin can prevent cell adhesion. New classes of drugs that blocks arteries could be identified using this invention.

### A. Materials and Methods

a) Fabrication of a Biosimulator:

A 10 cm plastic cell culture dish was used to fabricate the biosimulator. A sterile sharp stainless steel blade was used for etching. Etching was done in a laminar flow hood to maintain sterility. Different patterns were etched on the plastic surface.

b) Cell Culture:

Primary cells or hybridomas were cultured at a concentration of 250,000 cells per 10 cm biosimulator. The biosimulator was incubated at 37° C., with 5% CO2. On the third day the non-adherent cells formed distinct patterns in the biosimulator.

c) Cell Adhesion Inhibition:

The non-adherent cells (eg: hybridoma) was treated with salicylic acid or the adhesion inhibitor Pectasol (which prevents cancer metastasis) (Jiang et al. 2013).

### B. Results

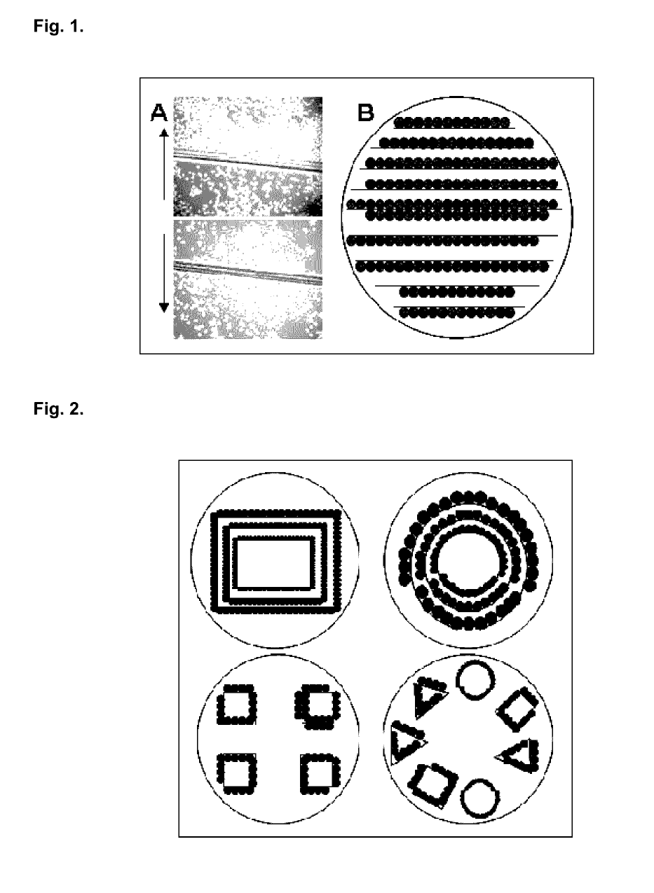

The non-adherent cells were cultured on an etched polystyrene biosimulator. There was no pattern formation on the first and second day of culture. After 3 days of culture the cells formed distinct pattern on the culture dish. All the cell lines tested (the hybridomas 4B7, 10D9, 1A10, 99D, Sp2/0, B56T) formed distinct patterns on the etched plastic surface. The pattern formation corresponded to the etched line on the plastic surface. When the biosimulator had etched horizontal lines, the non-adherent cells were seen on top of the etched line, whereas, on the lower half of the dish the non-adherent cells were below the etched line. Due to technical constraints to photograph a whole biosimulator under the microscope the cell alignment on the etched lines are shown graphically (FIG. 1). The cells were closely packed on the etched line. The experiment demonstrated that non-adherent cells could be converted to adherent cells and they could be induced to form distinct patterns on an etched surface. The pattern formation in non-adherent cells was found to be influenced by the etch design.

Cells were cultured on different etched designs. When the non-adherent cells were cultured on concentric squares/rectangles, the cells formed distinct patterns on the etched line. Whereas, when small squares were etched in the biosimulator, the cells were adhered on two sides inside and two sides outside the square. When concentric circles were etched the cells were adhered on the circle in the upper part of the circle, whereas on the lower half of the circle the cells adhered inside the circle. Similar patterns were also observed when small circles were etched on the edges of plastic dishes. When triangles were etched on edges of the dishes, the cells were always adhered to the inner two sides (FIG. 2). Based on these studies we also observed that cells have affinity to different sides when etching different shapes like spiral structures (Figure not shown). The phenomenon might be useful in designing probes for biomedical applications

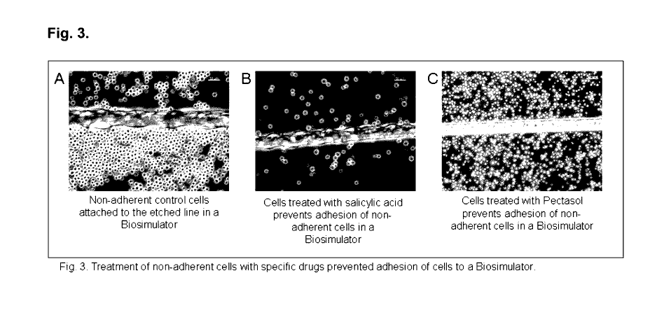

Salicylic acid is known to prevent cell-cell interaction and is used in animal models of diabetes (Cao et al. 2012), but its mechanism of action is not clearly known. When salicylic acid was treated with the non-adherent cells they inhibited cell adhesion to the etched surface (FIG. 3). The adhesion inhibitor Pectasol (which prevents cancer metastasis) was also used in our studies (Jiang et al. 2013). Treatment of non-adherent cells with Pectasol did not prevent cell proliferation, however, it prevented the cells to adhere to the plastic surface (FIG. 3C). The non-adherent cells lost the orientation property; the cells were found floating in the medium and did not have any affinity for the etched surface. The in vitro experiments with drugs to inhibit adhesion demonstrated that the phenomenon of pattern formation could be employed in drug discovery studies.

The study demonstrated that the non-adherent cells could be induced to form patterns in a biosimulator.

### REFERENCES

Cao Y, DuBois D C, Almon R R, Jusko W J. Biopharm Drug Dispos. 2012; 33:285-91.
Haycock J W. Methods Mol Biol. 2011; 695:1-15.
Jiang J, Eliaz I, Sliva D. Integr Cancer Ther. 2013; 12:145-152.
Leiva-Mora M, Alvarado-Cap\dot{o} Y, Acosta-Suárez M, Cruz-Martín M, Sánchez-García C, Roque B. Biotecnologia Vegetal 2010; 10: 49-52.
Lu S J, Feng Q, Park J S, Vida L, Lee B S, Strausbauch M, Wettstein P J, Honig G R, Lanza R. Blood. 2008; 112:4475-84.
Lu S J, Feng Q, Park J S, Lanza R. Methods Mol Biol. 2010; 636:105-21.
Ravi M, Paramesh V, Kaviya S R, Anuradha E, Solomon F D. J Cell Physiol. 2015; 230:16-26.
Rello S, Stockert J C, Moreno V, Gámez A, Pacheco M, Juarranz A, Cañete M, Villanueva A. Apoptosis. 2005; 10: 201-8.
Sarkar A, Kolitz S, Lauffenburger D A, Han J. Nat Commun. 2014; 5:3421.
Schindler R. Annu Rev Pharmacol. 1969; 9:393-406.
Stewart E J. J Bacteriol. 2012; 194:4151-60.
Turing, A. M. (1952). Philosophical Transactions of the Royal Society of London B 237 (641): 37-72.
Van Eelen W F. Industrial production of meat using cell culture methods. U.S. Pat. No. 7,270,829 B2.
Wang K, Steyn-Ross M L, Steyn-Ross D A, Wilson M T, Sleigh J W, Shiraishi Y. BMC Syst Biol. 2014; 8:45

1. An in vitro biosimulator comprising an etched plastic surface to induce adhesion and pattern formation of prokaryotic and eukaryotic non-adherent cells or their organelles.

2. The in vitro biosimulator is used for diagnosis of diseases based on the property of non-adherent cells to form distinct patterns.

3. The in vitro biosimulator is used for designing and engineering devices that resist biofouling.
